(19) United States
(12) Patent Application Publication
(10) Pub. No.: US 2025/0322903 A1
(43) Pub. Date: Oct. 16, 2025

(54) VISUALIZATIONS OF MOLECULAR SEQUENCES AND STRUCTURES

(71) Applicant: Benchling, Inc., San Francisco, CA (US)

(72) Inventors: Thomas Christiansen-Salameh, Oakland, CA (US); Andrew Yi Guo, Fremont, CA (US)

(21) Appl. No.: 19/175,981

(22) Filed: Apr. 10, 2025

Related U.S. Application Data
(60) Provisional application No. 63/632,273, filed on Apr. 10, 2024.

(51) Int. Cl. G16B 15/20; G16B 45/00

(52) U.S. Cl. CPC G16B 15/20; G16B 45/00

(57) ABSTRACT

Methods, systems, and apparatus, including computer programs encoded on a computer storage medium, for visualizing molecular sequences and structures. One of the methods includes receiving, from a sequence-oriented collection, data representing a sequence of molecular components, wherein the data specifies a different respective value for each of the molecular components in the sequence. Data representing an attachment molecule that is to be connected to one of the molecular components in the sequence is received from a structure-oriented collection. A user interface presentation that displays the sequence of molecular components using a different graphical representation for each molecular component of the sequence and that visually distinguishes a particular molecular component to which the attachment molecule is connected is generated.

### BACKGROUND

This specification relates to interfaces for visualizing molecules.

A molecule can refer to a group of bonded atoms. Examples of molecules include deoxyribonucleic acid (DNA) molecules, ribonucleic acid (RNA) molecules (e.g., messenger RNA) xeno nucleic acid (XNA) molecules, protein molecules, peptide molecules, antibody molecules, drug molecules, antibody-drug conjugate molecules, carbohydrate molecules, and lipid molecules. Other examples of molecules include oligonucleotides that are short DNA or RNA molecules having a wide range of applications in genetic testing, scientific research, and forensics. Examples of oligonucleotides include microRNA (miRNA), small interfering RNA (siRNA), small activating RNA (saRNA), antisense oligonucleotides (ASOs), and aptamers.

Computer-implemented systems and platforms for designing, representing, and working with molecules have traditionally been divided into two different domains: the “small molecule” domain and the “large molecule” domain.

The “small molecule” domain is primarily concerned with the atomic structure of a molecule or a compound. Chemically synthesized products, e.g., chemically synthesized drug products, are usually small-molecule products. The computer systems that support working with molecules in the small-molecule domain are thus mostly structure-oriented in the sense that the data is arranged to represent the atom-by-atom structures of the modules.

In contrast, the “large molecule” domain is primarily concerned with molecular sequences, e.g., protein sequences, antibody sequences, DNA sequences, and RNA sequences, to name just a few examples. The computer systems that support working with molecules in the large-molecule domain are thus mostly sequence-oriented in the sense that the data is arranged to represent sequences of elements.

The systems in these domains are largely incompatible and typically do not interact. Moreover, the underlying data is typically stored in separate and distinct database systems.

### SUMMARY

This specification describes a bioinformatics platform implemented as computer programs on one or more computers in one or more locations that can generate user interface presentations that merge aspects of structure-oriented and sequence-oriented systems. These technologies provide researchers with new capabilities for designing and working with modern molecular entities, which can require careful attention to both sequence-based and structure-based aspects of the molecular entities.

One example of a modern molecular entity that bridges the gap between small molecules and large molecules is small interfering RNA (siRNA). siRNA molecules are small strands of base pairs that interfere with the expression of specific genes by breaking down messenger RNA (mRNA) after transcription. These siRNA molecules work by attaching specific molecules to one end of them so that they can attach at particular locations of mRNA. When designing or synthesizing siRNA, it is thus particularly important for researchers to be able to visualize both the sequence of the siRNA molecule as well as the atomic structure of the attachment molecules.

Particular embodiments of the subject matter described in this specification can be implemented so as to realize one or more of the following advantages.

The bioinformatics platform described in this specification can generate user interface presentations that efficiently present both structure-oriented and sequence-oriented aspects of complex molecules. This functionality essentially merges data from two traditionally different domains, the small-molecule domain and the large-molecule domain. The techniques provide researchers with new capabilities to more efficiently work with and design very complex molecular compositions in a unified interface.

These techniques allow researchers to more easily compare structural variants. In such hybrid modalities, it is common to have many candidates that have a similar core structure but slightly vary in terms of the attached molecules, how they are connected, or what modifications exist on the core structure. In a system that can't present small molecules and large molecules in the same interface, it's difficult and cumbersome to inspect these kinds of variations at a glance. In contrast, using the techniques described in this specification, comparison between structures that are similar but vary slightly is far easier.

In addition, because the system can formally recognize each component of the structure, the structure as a whole, and the connections between each component at a molecular level, the ability to track results across experiments and analyze or visualize experimental data on such structures is greatly enhanced. This results in a much richer dataset of associations, as opposed to more crude approaches that can't model the structure as finely.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a diagram of an example bioinformatics platform.

FIG. 2 illustrates an example user interface presentation.

FIG. 3 is another example user interface presentation that can display both structure-oriented and sequence-oriented aspects of a molecule.

FIG. 4 is a flowchart of an example process for displaying both sequence-oriented and structure-oriented aspects of molecules

Like reference numbers and designations in the various drawings indicate like elements.

### DETAILED DESCRIPTION

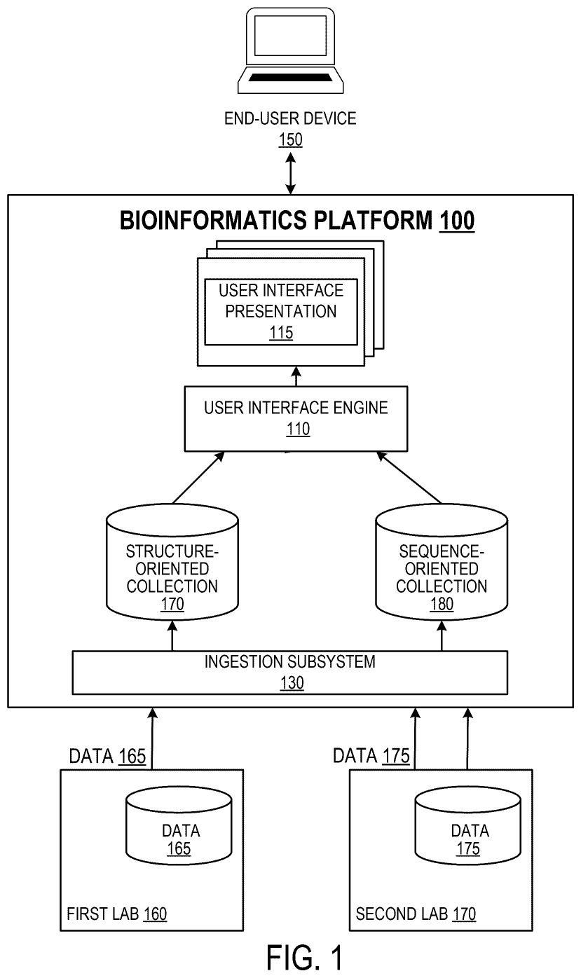

FIG. 1 is a diagram of an example bioinformatics platform 100 that can generate user interface presentations that merge aspects of structure-oriented and sequence-oriented collections. In other words, the platform 100 can generate user interface presentations that bridge the gap between large molecule and small molecule domains. The bioinformatics platform 100 is an example of a system implemented as computer programs on one or more computers in one or more locations in which the systems, components, and techniques described below are implemented.

The platform 100 makes use of data collections 170 and 180 from the two different domains.

The structure-oriented collection 170 is a collection of data for the small-molecule domain. The structure-oriented collection 170 can include data that represents structural relationships between atoms in a molecule or a compound. In the context of presenting molecular sequences along with structured-oriented information, a common example of a molecular entity whose atomic structure is important is chemical linkers that allow molecular components to be joined together. For example, a chemical linker can be used to join a molecular compound to a particular nucleotide in a sequence.

The sequence-oriented collection 180 is a collection of data for the large-molecule domain. The sequence-oriented collection 180 can include data representing molecular sequences, e.g., protein sequences, amino acid sequences, DNA sequences, and RNA sequences, to name just a few examples. The sequences can for example represent a molecule, e.g., deoxyribonucleic acid (DNA) molecule, a ribonucleic acid (RNA) molecule, an oligonucleotide, or any other appropriate molecule. For example, a naturally-occurring RNA molecule can be represented as a sequence of molecular nucleotides that each include three components: a sugar (e.g., ribose), a phosphate, and a nitrogenous base (e.g., guanine, uracil, adenine, or cytosine). Generally, the sequence data 165 can specify the sequence of molecular nucleotides in any appropriate format, e.g., using Hierarchical Editing Language for Macromolecules (HELM).

The collections 170 and 180 can be stored in separate database systems that each have a schema designed for their respective domains. Alternatively or in addition, the collections 170 and 180 can be stored in the same database in different relations or tables.

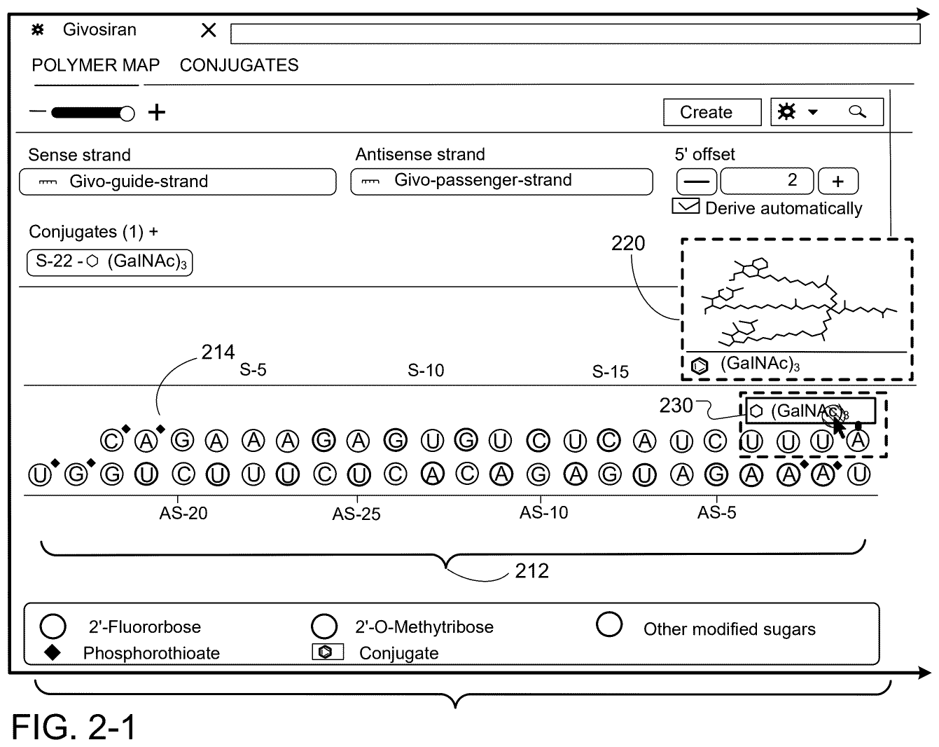

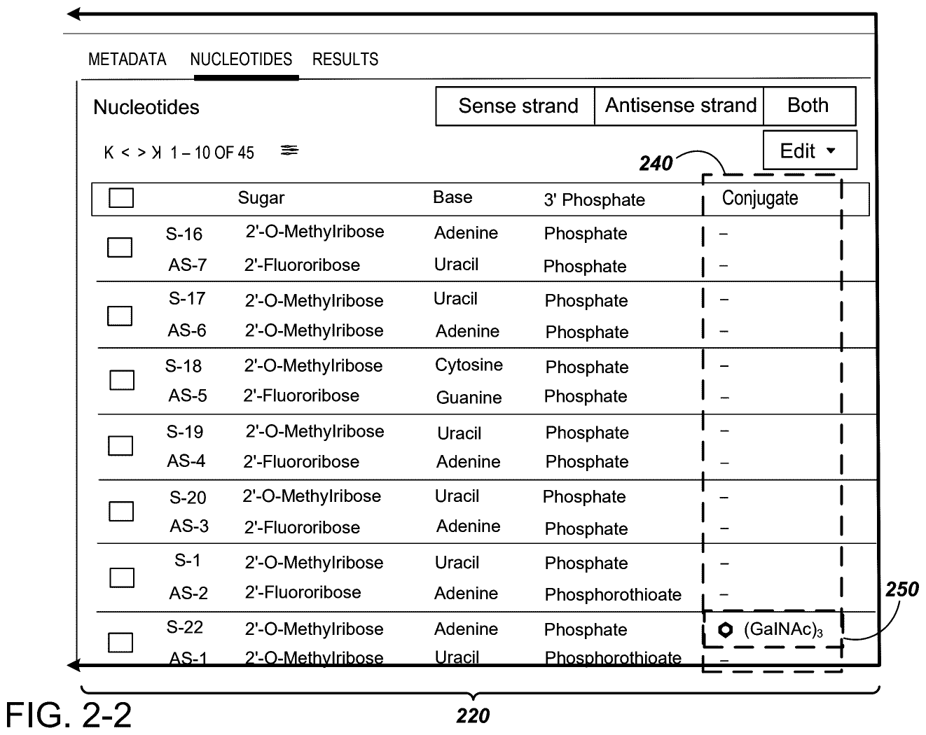

The user interface engine 110 can generate a user interface presentation 115 that presents both aspects of sequence-based and structure-based data in a unified presentation. As one example, the user interface presentation can display a molecular sequence annotated with attachment molecules that are linked to a particular unit of a of the molecular sequence using a chemical linker. In this specification, an attachment molecule is a molecular entity that can be attached at a particular attachment point of a molecular sequence. The molecular sequence can be a DNA sequence, an RNA sequence, or an amino acid sequence, to name just a few examples. Typically the structure of an attachment molecule is represented in a structure-oriented collection. Attachment molecules can include a chemical linker, a payload, or both. In some contexts, an attachment molecule may also be referred to as a conjugate or a bioconjugate to indicate what the final molecular product will represent after the attachment molecule is added to the sequence. An example user interface is described in more detail below with reference to FIG. 2.

The user interface engine 110 can also generate visual indications of chemical modifications to sequence entities. Various techniques for generating such user interface presentations are described in commonly owned U.S. patent application Ser. No. 17/939,667, which is herein incorporated by reference.

The bioinformatics platform 100 can provide the user interface presentation 115 for display to a user of the end-user device 150. Generally, the end-user device 150 can be an electronic device that is capable of requesting and receiving content over the network described above, e.g., the Internet. The end-user device 150 can include any appropriate client computing device such as a laptop/notebook computer, wireless data port, smart phone, personal data assistant (PDA), tablet computing device, one or more processors within these devices, or any other suitable processing device that can send and receive data over the network. For example, the end-user device 150 can include, e.g., a computer that includes an input device, such as a keypad, touch screen, or other device that can accept user information, and an output device that conveys information, including digital data, visual information, and/or the user interface presentation 115. The end-user 150 can include one or more client applications. A client application is any type of application that allows the end-user device 150 to request and view content on a respective client device. In some implementations, a client application can use parameters, metadata, and other information received, e.g., at launch, to access a particular set of data from the bioinformatics platform 100.

As described in more detail below with reference to FIG. 2, a user of the end-user device 150 can view the user interface presentation 115 and interact with the user interface presentation 115 using one or more controls presented in the user interface presentation 115. For example, the user can interact with the controls to view and select one or more attachment molecules for a particular sequence. After receiving the user selection, the user interface engine 110 can modify the user interface presentation 115 and can make corresponding modifications to the underlying data.

In some implementations, the platform 100 has an ingestion subsystem 130 that can ingest the structure-oriented collection 170, the sequence-oriented collection 180, or both from other systems. For example, these data collections can be stored in collections 165 and 175 in computer systems of respective labs 160 and 170. The ingestion subsystem 130 can then read and perform any appropriate conversions of the data in order to populate the collections 170 and 180. In this way, the platform 100 provides a powerful tool for researchers to easily combine and merge data from these different domains and to view this information in an integrated way.

FIG. 2 illustrates an example user interface presentation 200. The user interface presentation 200 is an example of a presentation that can be generated by the bioinformatics platform 100 of FIG. 1 to display both sequence-oriented and structure-oriented information in a combined interface.

The user interface presentation includes a sequence pane 210 and a list pane 210.

The sequence pane 210 graphically illustrates a molecular sequence 212. Each component of the molecular sequence is illustrated as having a corresponding letter or symbol. In this example, the molecular sequence is RNA, and thus the components of the sequence are A, C, G, and U. The bioinformatics platform can for example read a sequence or a portion of a sequence from a sequence-oriented database and generate a separate graphical element for each element of the sequence.

The sequence pane 210 also displays various modifications. For example, one of the elements of the sequence 212 has an modification annotation 214 indicating that the nucleotide at that position has had a chemical modification. A user can select the modification annotation 214, e.g., by clicking or mousing over the modification annotation 214, and the user interface presentation 200 can display more information about the modification. For example, the user interface presentation 200 can display structural information representing the modification to the nucleotide at that position.

The list pane 220 presents elements of the sequence 212 in list form. The list pane includes a number of columns that break down the components of each element of the molecular sequence 212. In this example, the components are a sugar, a base, and a phosphate. Other types of columns can also be used depending on the type of molecular sequence being displayed and the components thereof.

The list pane 220 also includes an attachment molecule column 240. The attachment molecule column 240 displays attachment molecules that are attached to various elements of the sequence. In this example, one element of the sequence is indicated as having an attachment molecule 250. The attachment molecule 250 is a N-acetylgalactosamine (GalNAc) molecule, which is a sugar molecule that can bind to cell proteins. GalNAc molecules are commonly used as attachment molecules for targeting particular therapeutic treatments.

The sequence pane 210 also displays an attachment molecule annotation 230. The attachment molecule annotation 230 is a graphical element that visually distinguishes an element of the sequence 212 in order to indicate that the element is linked to an attachment molecule. Thus, the sequence pane 210 can display different annotations for attachments and for chemical modifications.

In addition to visually representing that the element is linked to an attachment molecule, the sequence pane 210 can also generate a visual representation of the chemical structure of an attachment molecule. In this example, a user selection of the attachment molecule annotation 230 causes the user interface 200 to display a structure view 220. For example, the platform can obtain structural information about the attachment molecule from a structure-oriented database in order to generate the structure view 220. When a user selects the attachment molecule annotation, the user interface presentation can use the retrieved structural information to generate the structure view 220.

In some implementations, the user interface 200 allows users to directly modify the molecular sequence, the chemical structure of the attachment molecule, or both, within the user interface presentation 200. For example, a user can use the list pane 220 to select a particular molecular component of the sequence and to modify the attachment molecule to which the component is connected. For example, the system can retrieve multiple possible attachment molecules and allow the user to select a particular attachment molecule, e.g., from a pop-up window or a drop down menu. In some implementations, the user can edit the molecular sequence itself, e.g., by using the list pane to select different values for the sugar, base, phosphate, or some combination of these.

Thus, the user interface presentation 200 provides an easy way for users to modify and visualize both the sequence-oriented and structure-oriented aspects of a nucleotide sequence.

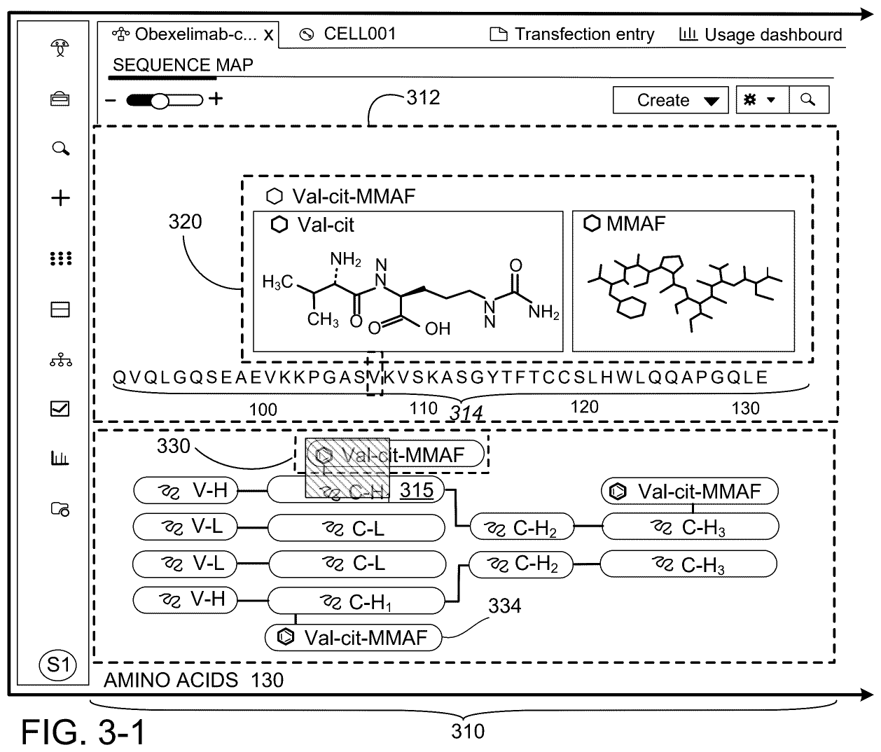

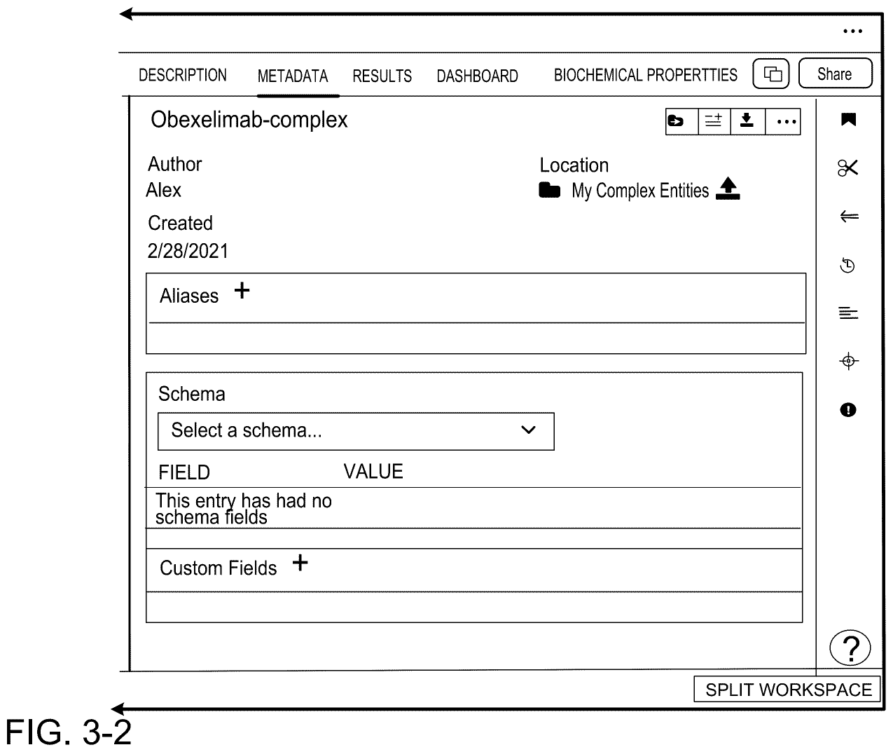

FIG. 3 is another example user interface presentation that can display both structure-oriented and sequence-oriented aspects of a molecule. In this example, the bioinformatics platform reads both structure-oriented and sequence-oriented data for a protein sequence 314. Common example of protein sequences include antibody-drug conjugates (ADCs). ADCs are commonly designed for providing particular therapeutic treatments, e.g., for cancer treatments. An ADC can be formed by attaching a payload to an antibody complex using a linking molecule. Thus, an ADC can be formed by an antibody and an attachment molecule.

In this context, the primary aspect of the antibody is an amino acid sequence, while the primary aspect of the attachment molecule is its atomic structure. The example user interface presentation 300 is an example that allows a bioinformatics platform to display both the sequence-oriented and structure-oriented aspects of an ADC.

The example user interface presentation 300 includes a protein overview pane 310 that displays an overview of twelve distinct protein sequence chains of an antibody and how they are connected.

The user interface presentation 300 also includes a protein sequence pane 312 that displays an amino acid sequence 314. For each position in the amino acid sequence 314, the protein sequence pane 312 displays a respective graphical representation, e.g., a different character, for each amino acid in the amino acid sequence 314.

When a user selects any of the protein sequences in the protein overview pane 310, the protein sequence pane 312 updates to display the corresponding amino acid sequence. Thus in this example, a user has selected the protein sequence 315 in the protein overview pane 310, and the protein sequence pane 312 displays the amino acid sequence 314.

The example ADC has three attachment molecules 330, 332, and 334. As described above, each attachment molecule can include a linker, a payload, or both. The protein overview pane 310 graphically represents the protein sequences to which the attachment molecules are connected.

When a user selects the graphical representation of the attachment molecule or a protein sequence having an attachment molecule, the system can generate a chemical structure pane 320 that displays a graphical representation of the chemical structure of the attachment molecule. In addition, the protein sequence pane can visually distinguish the amino acid in the amino acid sequence to which the attachment molecule is connected.

In this example, the structure pane 320 separately displays a linking molecule and a payload. In this case, the linking molecule is a valine-citrulline dipeptide, and the payload is a cytotoxic payload known as monomethyl auristatin F (MMAF).

As can be seen from this example, the user interface presentation allows a user to easily and more efficiently obtain and view sequence-oriented and structure-oriented aspects of complex bioconjugates. In one single user interface presentation, a user can view the overall structure of the bioconjugate using the protein overview pane 310, the attachment points for the attachment molecules in the overall structure, the amino acid sequences using the protein sequence pane, and the atomic structure of the attachment molecules, including the atomic structure of linking molecules and corresponding payloads. Conventional systems would typically store the sequence-oriented aspects of an antibody and the structure-oriented aspects of the payload in different systems that are incompatible and that do not interact.

As described above, the user interface presentation 300 also provides users with the capabilities to easily modify aspects of the molecular complex. For example, the system can provide the user with selection options for selecting a different chemical linker or a different payload within or alongside the structure pane 320. In some implementations, the user can also select and add attachment molecules to protein sequences in the protein overview pane 310. When the user adds an attachment molecule to a particular protein sequence, the system can prompt the user to specify a particular molecular component within the sequence to which the attachment molecule will be connected. The system can then visually distinguish that selected molecular component, e.g., with an attachment annotation, so that it is clear which component within the sequence is connected to the attachment molecule.

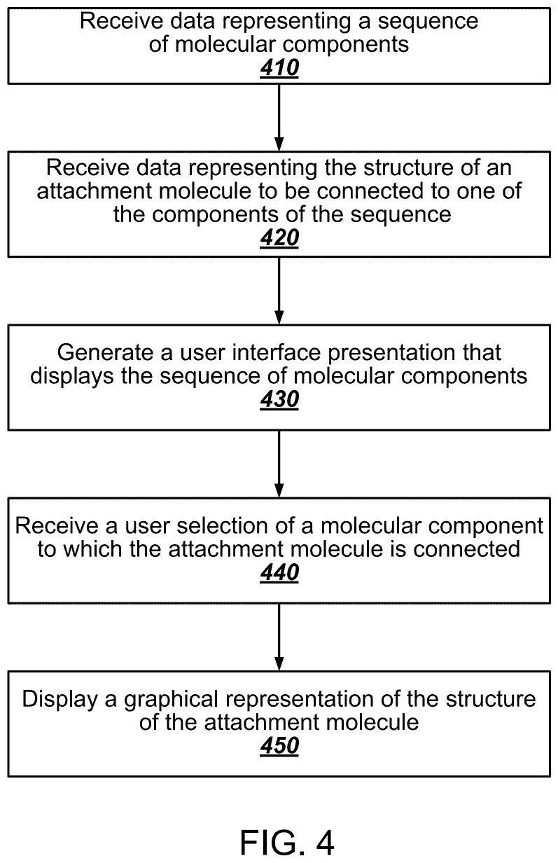

FIG. 4 is a flowchart of an example process for displaying both sequence-oriented and structure-oriented aspects of molecules. The example process can be performed by a bioinformatics platform implemented as one or more computers in one or more locations and programmed in accordance with this specification. The example process will be described as being performed by a system of one or more computers.

The system receives data representing a sequence of molecular components (410). For example, the system can obtain the sequence from a sequence-oriented database as described above. Generally, the data will specify a single respective value for each of the molecular components in the sequence. The sequence can be any appropriate molecular sequence, e.g., a nucleotide sequence or an amino acid sequence.

The system receives data representing the structure of an attachment molecule that is to be connected to one of the molecular components in the sequence (420). As described above, the structure of an attachment molecule can be represented in a structure-oriented database. The data representing the attachment molecule can include a chemical linker, a payload, or both.

The system generates a user interface presentation that displays the sequence of molecular components and that visually distinguishes a particular molecular component to which the attachment molecule is connected (430). In other words, a user of the user interface presentation can by visual inspection see which of the molecular components is connected to an attachment molecule. In this context, being connected to a molecule refers to the design of the molecule using a bioinformatics platform. Actual molecular connections in a laboratory need not have been formed. In some implementations, the system displays an attachment annotation to indicate which component of the molecular sequence is connected to the attachment molecule. The attachment annotation can display a name of the attachment molecule.

The system receives a user selection corresponding to the particular molecular component of the sequence to which the attachment molecule is connected (440). The user selection can be any appropriate selection mechanism, e.g., a tap or a long press on a touch-sensitive display, hovering over or clicking with a mouse, selecting a key on a keyboard, or any other appropriate user selection mechanism.

The system displays a graphical representation of the structure of the attachment molecule connected to the sequence of molecular components (450). In some implementations, the system displays the structure of the attachment molecule alongside the sequence of molecular components so that both the structure-oriented and sequence-oriented aspects of the molecule are displayed in one unified interface.

This allows the system to simultaneously display information about the molecule at multiple levels of granularity. For example, in the context of a protein sequence, the system can simultaneously display an overview of the entire protein complex, the components of a particular protein sequence within the protein complex, as well as the atomic structure of an attachment molecule and where that attachment molecule is connected to the protein sequence.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A computer-implemented method comprising:
receiving, from a sequence-oriented collection, data representing a sequence of molecular components, wherein the data specifies a different respective value for each of the molecular components in the sequence;
receiving, from a structure-oriented collection, data representing an attachment molecule that is to be connected to one of the molecular components in the sequence;
generating a user interface presentation that displays the sequence of molecular components using a different graphical representation for each molecular component of the sequence and that visually distinguishes a particular molecular component to which the attachment molecule is connected;
receiving a user selection corresponding to the particular molecular component of the sequence to which the attachment molecule is connected; and
in response, displaying, within the user interface presentation, a graphical representation of the structure of the attachment molecule connected to the sequence of molecular components.

2. The method of claim 1, wherein the user interface presentation visually distinguishes the particular molecule component using an attachment annotation.

3. The method of claim 2, wherein the attachment annotation lists a name of the attachment molecule.

4. The method of claim 2, wherein the user selection is a user selection of the attachment annotation.

5. The method of claim 1, wherein the sequence of molecular components is one protein sequence of a protein complex having a plurality of protein complexes.

6. The method of claim 5, wherein the user interface presentation displays a protein overview of the plurality of protein complexes.

7. The method of claim 6, wherein the user selection that causes displaying the graphical representation of the structure of the attachment molecule is a selection of the protein sequence within the protein overview.

8. The method of claim 7, wherein the protein overview visually distinguishes protein sequences having attachment molecules.

9. The method of claim 8, wherein the protein overview displays a name of each attachment molecule for each protein sequence having an attachment molecule.

10. The method of claim 9, wherein the sequence is an amino acid sequence, and wherein the user interface presentation visually distinguishes a particular amino acid in the amino acid sequence to which the attachment molecule is connected.

11. The method of claim 10, wherein the graphical representation of the structure of the attachment molecule comprises a graphical representation of a structure of a linking molecule and a graphical representation of a structure of a payload.

12. A system comprising:
one or more computers and one or more storage devices storing instructions that are operable, when executed by the one or more computers, to cause the one or more computers to perform operations comprising:
receiving, from a sequence-oriented collection, data representing a sequence of molecular components, wherein the data specifies a different respective value for each of the molecular components in the sequence;
receiving, from a structure-oriented collection, data representing an attachment molecule that is to be connected to one of the molecular components in the sequence;
generating a user interface presentation that displays the sequence of molecular components using a different graphical representation for each molecular component of the sequence and that visually distinguishes a particular molecular component to which the attachment molecule is connected;
receiving a user selection corresponding to the particular molecular component of the sequence to which the attachment molecule is connected; and
in response, displaying, within the user interface presentation, a graphical representation of the structure of the attachment molecule connected to the sequence of molecular components.

13. The system of claim 12, wherein the user interface presentation visually distinguishes the particular molecule component using an attachment annotation.

14. The system of claim 13, wherein the attachment annotation lists a name of the attachment molecule.

15. The system of claim 13, wherein the user selection is a user selection of the attachment annotation.

16. The system of claim 12, wherein the sequence of molecular components is one protein sequence of a protein complex having a plurality of protein complexes.

17. The system of claim 16, wherein the user interface presentation displays a protein overview of the plurality of protein complexes.

18. The system of claim 17, wherein the user selection that causes displaying the graphical representation of the structure of the attachment molecule is a selection of the protein sequence within the protein overview.

19. The system of claim 18, wherein the protein overview visually distinguishes protein sequences having attachment molecules.

20. One or more non-transitory computer storage media encoded with computer program instructions that when executed by one or more computers cause the one or more computers to perform operations comprising:
receiving, from a sequence-oriented collection, data representing a sequence of molecular components, wherein the data specifies a different respective value for each of the molecular components in the sequence;
receiving, from a structure-oriented collection, data representing an attachment molecule that is to be connected to one of the molecular components in the sequence;
generating a user interface presentation that displays the sequence of molecular components using a different graphical representation for each molecular component of the sequence and that visually distinguishes a particular molecular component to which the attachment molecule is connected;
receiving a user selection corresponding to the particular molecular component of the sequence to which the attachment molecule is connected; and
in response, displaying, within the user interface presentation, a graphical representation of the structure of the attachment molecule connected to the sequence of molecular components.
